(19) United States
(12) Patent Application Publication
(10) Pub. No.: US 2009/0088647 A1
(43) Pub. Date: Apr. 2, 2009

(54) ULTRASONIC ENDOSCOPE

(75) Inventors: Kazuhiko Nagano, Kaisei-machi (JP); Hiroaki Hyuga, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/201,538

(22) Filed: Aug. 29, 2008

(30) Foreign Application Priority Data
Sep. 28, 2007 (JP) 2007-254953

(51) Int. Cl. A61B 8/14

(52) U.S. Cl. 600/463

(57) ABSTRACT

An ultrasonic endoscope capable of suppressing temperature rise of an insertion part without increase in diameter. The ultrasonic endoscope includes: an ultrasonic transducer part having plural ultrasonic transducers; an exterior member for holding the ultrasonic transducer part; an opening formed in the exterior member; a heat conducting member arranged inside of the exterior member and connected to the ultrasonic transducer part; and a heat radiating member provided on an outer surface of the exterior member and connected to the heat conducting member via the opening. For example, a part of the heat radiating member is located within the opening and the part is connected to the heat conducting member. The heat radiating member is electrically insulated from the ultrasonic transducer part.

### BACKGROUND OF THE INVENTION

1. Field of the Invention

The present invention relates to an ultrasonic endoscope to be used for body cavity examination of upper digestive organs, bronchial tube, and so on.

2. Description of a Related Art

In medical fields, various imaging technologies have been developed in order to observe the interior of an object to be inspected and make diagnoses. Among them, especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but also gynecology, circulatory system, digestive system, etc.

The ultrasonic imaging is an image generation technology utilizing the nature of ultrasonic waves that the waves are reflected at a boundary between regions with different acoustic impedances (e.g., a boundary between structures) Typically, an ultrasonic diagnostic apparatus using ultrasonic imaging is provided with a body surface ultrasonic probe to be used in contact with the object or an intracavity ultrasonic probe to be used by being inserted into a body cavity of the object. Further, in recent years, an ultrasonic endoscope in combination of an endoscope for optically observing the interior of the object and an ultrasonic probe for intracavity has been used.

Ultrasonic beams are transmitted toward the object such as a human body and ultrasonic echoes generated in the object are received by using the ultrasonic endoscope, and thereby, ultrasonic image information is acquired. On the basis of the ultrasonic image information, ultrasonic images of structures (e.g., internal organs, diseased tissues, or the like) existing within the object are displayed on a display unit of an ultrasonic endoscopic apparatus main body connected to the ultrasonic endoscope.

As an ultrasonic transducer for transmitting and receiving ultrasonic waves, a vibrator (piezoelectric vibrator) having electrodes formed on both sides of a material that expresses a piezoelectric property (a piezoelectric material) is generally used. When a voltage is applied to the electrodes of the vibrator, the piezoelectric material expands and contracts due to the piezoelectric effect and generates ultrasonic waves. Accordingly, plural vibrators are one-dimensionally or two-dimensionally arranged and the vibrators are sequentially driven, and thereby, an ultrasonic beam to be transmitted in a desired direction can be formed. Further, the vibrators expand and contract by receiving propagating ultrasonic waves and generate electric signals. These electric signals are used as reception signals of the ultrasonic waves.

When ultrasonic waves are transmitted, drive signals having great energy are supplied to the ultrasonic transducers. In this regard, not the entire energy of the drive signals is converted into acoustic energy but a significant proportion of the energy becomes heat, and there has been a problem that the temperature rises in use of the ultrasonic endoscope. However, the insertion part of the ultrasonic endoscope is used in direct contact with the living body such as a human body, and a request that the surface temperature of the insertion part of the ultrasonic endoscope is controlled to a predetermined temperature or less has been made for safety reasons of low-temperature burn and so on.

As a related technology, Japanese Patent Application Publication JP-A-9-140706 discloses a technology of collecting heat generated from a heat source within a probe by using heat collecting means within the probe and guiding the heat collected by the heat collecting means to a location apart from the heat source by using heat transfer means such as a heat pipe. However, the outer diameter of the ultrasonic probe needs to be smaller in the case where the ultrasonic probe is inserted into a human body, while the diameter of the heat transfer means such as a heat pipe needs to be larger for sufficiently high heat transfer coefficient of the heat transfer means. Accordingly, it is difficult to apply the technology of JP-A-9-140706 to an ultrasonic endoscope.

Japanese Patent Application Publication JP-P2006-204552A discloses a technology of cooling a vibrator part by transferring the heat generated in the vibrator part and a circuit board to a shield case via a heat conducting part, and allowing the heat transferred to the shield case to be absorbed by a heat absorbing part including a refrigerant feeder and a refrigerant pipe. However, the outer diameter of the ultrasonic probe needs to be smaller in the case where the ultrasonic probe is inserted into a human body, and it is difficult to apply the technology of JP-P2006-204552A to an ultrasonic endoscope to be inserted into a human body.

Japanese Registered Utility Model JP-Z-3061292 discloses that a heat transfer structure is provided in contact with an integrated circuit within an ultrasonic transducer for extracting heat generated there to the outside, and the heat extracted by the heat transfer structure is transferred to a conducting material that functions as a heat sink within a communication cable. However, in an ultrasonic endoscope, the signal cable has a small sectional area, and, if the signal cable is used for heat dissipation, no sufficient heat dissipation effect is obtained due to the small sectional area.

### SUMMARY OF THE INVENTION

The present invention has been achieved in view of the above-mentioned problems. A purpose of the present invention is to provide an ultrasonic endoscope capable of suppressing temperature rise without increase in diameter.

In order to accomplish the purpose, an ultrasonic endoscope according to one aspect of the present invention includes: an ultrasonic transducer part having plural ultrasonic transducers; an exterior member for holding the ultrasonic transducer part; an opening formed in the exterior member; a heat conducting member arranged inside of the exterior member and connected to the ultrasonic transducer part; and a heat radiating member provided on an outer surface of the exterior member and connected to the heat conducting member via the opening.

According to the present invention, the heat generated in the ultrasonic transducer part transfers to the heat radiating member provided on the outer surface of the exterior member via the heat conducting member, and released to the outside from the heat radiating member. Thus, since the heat radiating member is provided on the outer surface of the exterior member, the temperature rise of the ultrasonic endoscope can be suppressed without increase in diameter.

### BRIEF DESCRIPTION OF THE DRAWINGS

FIG. 1 is a schematic diagram showing an appearance of an ultrasonic endoscope according to the respective embodiments of the present invention;

FIG. 2 is a perspective view showing a leading end of the ultrasonic endoscope according to the first embodiment;

FIG. 3 is a sectional view showing a structure of a leading end of an insertion part of the ultrasonic endoscope according to the first embodiment;

FIG. 4 is a perspective view for explanation of a configuration of an ultrasonic transducer;

FIG. 5 is a sectional view for explanation of a configuration of an ultrasonic endoscope according to the second embodiment;

FIG. 6 is a sectional view for explanation of a configuration of an ultrasonic endoscope according to the third embodiment;

FIG. 7 is a sectional view for explanation of a configuration of an ultrasonic endoscope according to the fourth embodiment; and

FIG. 8 shows an ultrasonic endoscopic apparatus including the ultrasonic endoscope and an ultrasonic endoscopic apparatus main body according to the respective embodiments of the present invention.

### DESCRIPTION OF THE PREFERRED EMBODIMENTS

Hereinafter, embodiments of the present invention will be explained in detail with reference to the drawings. The same reference numbers will be assigned to the same component elements and the description thereof will be omitted.

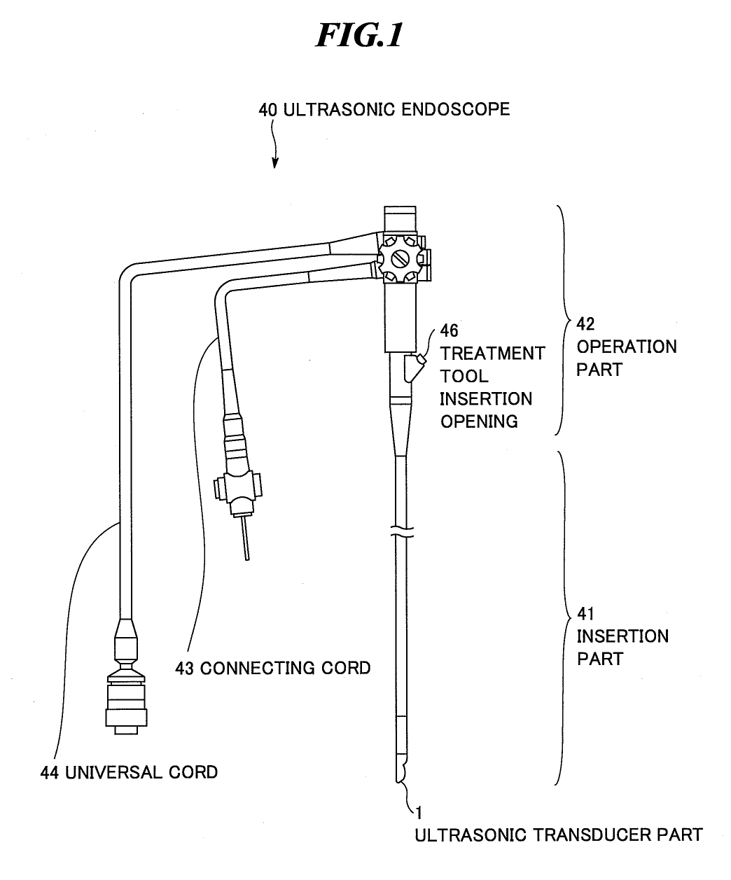

FIG. 1 is a schematic diagram showing an appearance of an ultrasonic endoscope according to the respective embodiments of the present invention. As shown in FIG. 1, an ultrasonic endoscope 40 includes an insertion part 41, an operation part 42, a connecting cord 43, and a universal cord 44. The insertion part 41 includes an elongated tube formed of a member having flexibility for insertion into the body (e.g., into the bronchial tube) of an object to be inspected, and an ultrasonic transducer part 1 at the leading end thereof.

The operation part 42 is provided at the base end of the insertion part 41 and connected to an ultrasonic endoscopic apparatus main body (not shown) via the connecting cord 43 and the universal cord 44. A treatment tool insertion opening 46 provided in the operation part 42 is a hole for leading in a treatment tool such as a punctuation needle or forceps. Various treatments are performed within a body cavity of an object to be inspected by operating it with the operation part 42.

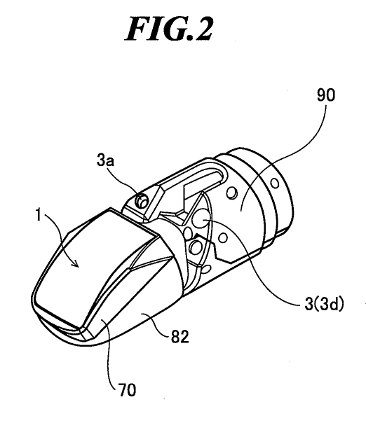

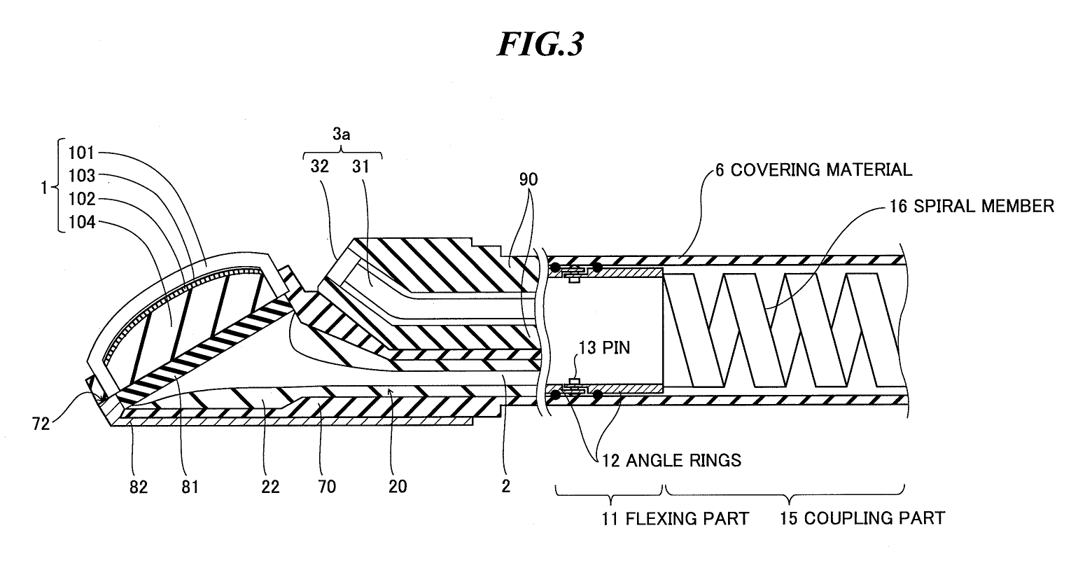

FIG. 2 is a perspective view showing the leading end of the insertion part of the ultrasonic endoscope according to the first embodiment of the present invention. FIG. 3 is a sectional view showing a structure of the leading end of the insertion part of the ultrasonic endoscope according to the first embodiment. As shown in the drawings, the leading end of the insertion part of the ultrasonic endoscope according to the embodiment has an ultrasonic transducer part 1 for transmitting and receiving ultrasonic waves, signal lines 2 for transmitting signals between the ultrasonic transducer part 1 and the ultrasonic endoscopic apparatus main body, a light guide part 3a for applying light to an affected part, an imaging part 3 (shown in FIG. 2) for optically imaging the affected part, an exterior member 70 for holding the ultrasonic transducer part 1 and the leading end of the signal lines 2, an optics holding member 90 attached to the exterior member 70 and for holding the imaging part 3 and the light guide part 3a, a flexing part 11 flexible for supporting the exterior member 70 and the optics holding member 90, a coupling part 15 for coupling the flexing part 11 to the operation part 42 (shown in FIG. 1) , and a covering material 6 for covering at least the flexing part 11 and the coupling part 15. The outer diameter of the leading end is φ6.9 mm or less, for example. The exterior member 70 and the optics holding member 90 are formed of a resin such as polyetherimide, for example.

The imaging part 3 has an observation window 3d provided in the optics holding member 90, an objective lens fit in the observation window 3d, and an input end of a solid-state image sensor such as a CCD camera or an image guide provided in the imaging position of the objective lens. The light guide part 3a has an illumination window 32 provided in the optics holding member 90 and an optical fiber 31 for outputting light from the illumination window 32. An illumination lens is fit in the illumination window 32.

The flexing part 11 is configured by arranging points of support for bending of plural top-like angle rings 12 with displacement of 90° with respect to each other in a staggered manner. The top-like angle rings 12 are connected to one another so as to be relatively displaced by pins 13 and form a hinge structure. The coupling part 15 includes a spiral member 16. The spiral member 16 is generally formed of stainless steel, for example. The covering material 6 is formed of an electrically insulating material of fluorine-containing rubber, for example.

The ultrasonic transducer part 1 is a convex-type multirow array, for example, and has plural ultrasonic transducers 102 provided on the upper face of a backing material 104, and an acoustic lens 101 covering the plural ultrasonic transducers 102, for example. One or some acoustic matching layers 103 are provided between the acoustic lens 101 and the ultrasonic transducers 102.

The acoustic matching layer 103 is formed of Pyrex (registered trademark) glass or an epoxy resin containing metal powder, which easily propagates ultrasonic waves for providing matching of acoustic impedances between the object as a living body and the ultrasonic transducers 102. Thereby, the ultrasonic waves transmitted from the ultrasonic transducers 102 efficiently propagate within the object.

The acoustic lens 101 is exposed from the upper surface of the exterior member 70, and formed of silicone rubber, for example. The acoustic lens 101 focuses an ultrasonic beam transmitted from the ultrasonic transducers 102 and propagating through the acoustic matching layer 103 at a predetermined depth within the object.

The backing material 104 is formed of an elastomer such as rubber, for example, or may include mixture of a base material formed of an elastomer and a filler having higher heat conductivity than the base material. In this case, as the filler, ferrite, tungsten, alumina, or the like is used. The ultrasonic transducer part 1 is housed within the exterior member 70 with the acoustic lens 101 exposed. Since the ultrasonic waves generated by the ultrasonic transducers 102 are also applied to the backing material 104, heat is generated from the backing material 104.

A heat conducting member 81 is connected to the back face of the backing material 104. The heat conducting member 81 is a plate-like member, for example, and provided along a direction intersecting the side surface of the exterior member 70, for example, diagonally provided relative to the inner surface of the exterior member 70. The thickness of the heat conducting member 81 is, for example, from 30 μm to 1 mm, especially preferably, from 500 μm to 700 μm. It is preferable that the heat conducting member 81 is connected to the entire surface of the back face of the backing material 104, but may be connected to a part (e.g., more than a half of the back face) thereof. The heat conducting member 81 includes an electrically insulating material having a coefficient of thermal conductivity equal to or more than 10 W/(m·K) such as aluminumnitride (AlN), for example. A part, for example, a side surface of the heat conducting member 81 faces an opening 72 formed in the exterior member 70. The opening 72 is formed from the rear surface of the exterior member 70 to the side lower part (e.g., the lower half part). The heat conducting member 81 and the backing material 104 are connected via an adhesive having high thermal conductivity, for example.

A heat radiating member 82 is attached to the outer surface of the side of the exterior member 70. The heat radiating member 82 is a plate-like member along the outer surface of the exterior member 70, and formed of a material having a coefficient of thermal conductivity equal to or more than 10 W/(m·K), for example, stainless steel (e.g., SUS 304). The heat radiating member 82 may be thin, and its thickness is from 0.1 mm to 0.2 mm, for example. The heat radiating member 82 covers the opening 72 formed on the side surface of the exterior member 70 to seal it, and a part of the heat radiating member enters the opening 72. The part is connected to the heat conducting member 81. The heat conducting member 81 and the heat radiating member 82 are connected to each other via an adhesive having high thermal conductivity, for example. The end face of the part entering the opening 72 of the exterior member 70 is flush with the inner surface of the exterior member 70, for example. The heat radiating member 82 is provided from the rear surface of the exterior member 70 to the side lower part (e.g., the lower half part), for example, but may be provided on the entire surface of the side. In the former case, the area of the part where the ultrasonic transducers 102 are provided can be made larger. According to a simulation, when the coefficient of thermal conductivity of the heat conducting member 81 is 10 W/mK in the embodiment, for example, the temperature rise of the surface of the acoustic lens 101 can be reduced by 25% compared to the case without the heat conducting member 81 or the heat radiating member 82.

The signal lines 2 include plural shield lines connected to the plural ultrasonic transducers 102, respectively, for example. The signal lines 2 pass through a signal line holding part 20. The leading end of the signal line holding part 20 is connected to the heat conducting member 81 and a part of the signal line holding part 20 is in contact with the exterior member 70. The interior of the signal line holding part 20 is filled with a heat conducting filling material 22. The heat conducting filling material 22 has a coefficient of thermal conductivity equal to or more than 2 W/(m·K) and an electrically insulation property such as a silicone rubber adhesive KE-3467, KE-1867, or KE-32-2152 manufactured by Shin-Etsu Chemical Co., Ltd., for example. The coefficient of thermal conductivity of the heat conducting filling material 22 is more preferably equal to or more than 10 W/(m·K).

In the above-mentioned configuration, the heat generated in the ultrasonic transducers 102 transfers to the heat conducting member 81 via the backing material 104, and the heat generated in the backing material 104 transfers to the heat conducting member 81. The heat that has transferred to the heat conducting member 81 transfers to the heat radiating member 82 via the part entering the opening 72 and released to the outside from the heat radiating member 82. Therefore, the heat staying inside the exterior member 70 is suppressed, and consequently, the temperature rise at the leading end of the insertion part of the ultrasonic endoscope 40 can be suppressed. When a filler having high heat conductivity is mixed in the backing material 104, the effect becomes especially great. Further, since the heat conducting member 81 is formed of an electrically insulating material, the insulation of the ultrasonic transducer part 1 from the heat radiating member 82 can be ensured.

Further, it is not necessary to increase the diameter of the leading end of the insertion part of the ultrasonic endoscope 40 for providing the heat conducting member 81, and the heat radiating member 82 may be thin. Therefore, the diameter of the leading end of the insertion part of the ultrasonic endoscope 40 is not increased.

Furthermore, since the signal line holding part 20 is filled with the heat conducting filling material 22, the heat generated in the ultrasonic transducers 102 also transfers to the heat conducting filling material 22 via the backing material 104, and further released to the other part (e.g., the exterior member 70) via the heat conducting filling material 22. Therefore, the temperature rise at the leading end of the insertion part of the ultrasonic endoscope 40 can be further suppressed.

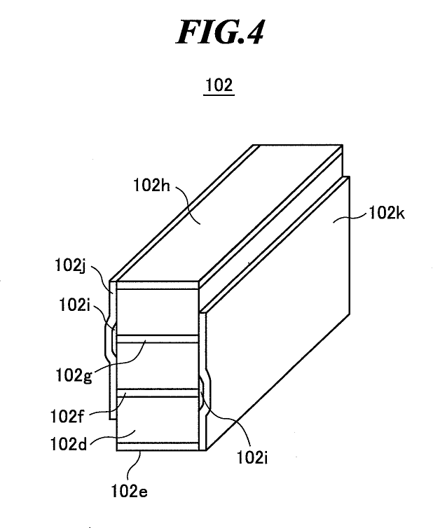

FIG. 4 is a perspective view for explanation of the configuration of the ultrasonic transducer 102. The ultrasonic transducer 102 includes plural piezoelectric material layers 102d formed of PZT or the like, a lower electrode layer 102e, internal electrode layers 102f and 102g alternately inserted between the plural piezoelectric material layers 102d, an upper electrode layer 102h, electrically insulating films 102i, and side electrodes 102j and 102k.

The lower electrode layer 102e is connected to the side electrode 102k at the right side in the drawing and electrically insulated from the side electrode 102j at the left side in the drawing. The upper electrode layer 102h is connected to the side electrode 102j and electrically insulated from the side electrode 102k. Further, the internal electrode layer 102f is connected to the side electrode 102j and electrically insulated from the side electrode 102k by the electrically insulating film 102i. On the other hand, the internal electrode layer 102g is connected to the side electrode 102k and electrically insulated from the side electrode 102j by the electrically insulating film 102i. The plural electrodes of the ultrasonic transducer 102 are formed in this fashion, three pairs of electrodes for applying electric fields to the three layers of piezoelectric material layers 102d are connected in parallel. The number of piezoelectric material layers is not limited to three, but may be two or four or more.

In the multilayered ultrasonic transducer 102, the area of electrodes in contact with the piezoelectric material layers 102d is larger than that of a single-layered element, and the electric impedance is lower. Therefore, the multilayered ultrasonic transducer has increased vibration output and operates more efficiently for the applied voltage than the single-layered piezoelectric vibrator having the same size. Specifically, given that the number of piezoelectric material layers 102d is N, the number of the piezoelectric material layers is N-times the number of the single-layered piezoelectric vibrator and the thickness of each piezoelectric material layer is 1/N of that of the single-layered piezoelectric vibrator, and the electric impedance of the ultrasonic transducer 102 is 1/N²-times. Therefore, the electric impedance of ultrasonic transducer 102 can be adjusted by increasing or decreasing the number of stacked piezoelectric material layers 102d, and thus, the electric impedance matching between a drive circuit or preamplifier and itself is easily provided, and the sensitivity can be improved.

On the other hand, the capacitance is increased due to the stacked form of the ultrasonic transducer 102, and the amount of heat generated from the ultrasonic transducer 102 becomes larger. However, since the heat conducting member 81 and the heat radiating member 82 are provided in the embodiment, the heat generated in the ultrasonic transducers 102 is efficiently released to the outside, and consequently, the temperature rise at the leading end of the insertion part of the ultrasonic endoscope 40 can be suppressed.

As described above, according to the first embodiment of the present invention, the heat generated in the ultrasonic transducers 102 transfers to the heat conducting member 81 via the backing material 104. The heat that has transferred to the heat conducting member 81 transfers to the heat radiating member 82 attached to the outer surface of the side of the exterior member 70 via the part entering the opening 72 and is released to the outside from the heat radiating member 82. Thus, since the heat radiating member 82 is attached to the outer surface of the exterior member 70, the temperature rise at the leading end of the insertion part of the ultrasonic endoscope 40 can be suppressed even when the diameter of the insertion part is small. Further, since the conducting member 81 is provided along the direction intersecting the side surface of the exterior member 70, if the sectional area of the heat conducting member 81 is increased for raising the heat conduction efficiency of the heat conducting member 81, the diameter of the insertion part of the ultrasonic endoscope 40 is not increased. Furthermore, the heat conducting member 81 is formed of an electrically insulating material, the insulation of the heat radiating member 82 located on the outer surface of the exterior member 70 from the ultrasonic transducer part 1 can be ensured.

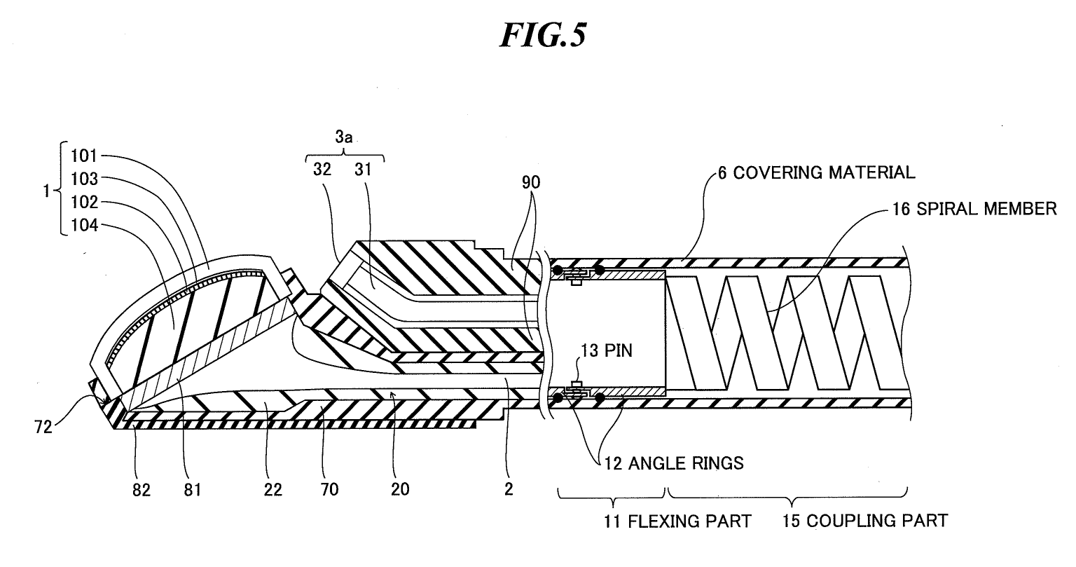

FIG. 5 is a sectional view for explanation of a configuration of an ultrasonic endoscope according to the second embodiment of the present invention, and corresponds to FIG. 3 in the first embodiment. The ultrasonic endoscope according to the embodiment has the same configuration as that of the ultrasonic endoscope according to the first embodiment except that the heat conducting member 81 is formed of a conducting material (e.g., stainless steel such as SUS 304) and the heat radiating member 82 is formed of an electrically insulating material. Each of the heat conducting member 81 and the heat radiating member 82 preferably has a coefficient of thermal conductivity equal to or more than 10 W/(m·K).

Also according to the embodiment, the temperature rise at the leading end of the insertion part of the ultrasonic endoscope 40 can be suppressed as is the case of the first embodiment, and the increase in the diameter of the leading end of the ultrasonic endoscope 40 can be suppressed. Further, since the heat radiating member 82 is formed of an electrically insulating material, the insulation of the heat radiating member 82 located on the outer surface of the exterior member 70 from the ultrasonic transducer part 1 can be ensured as is the case of the first embodiment.

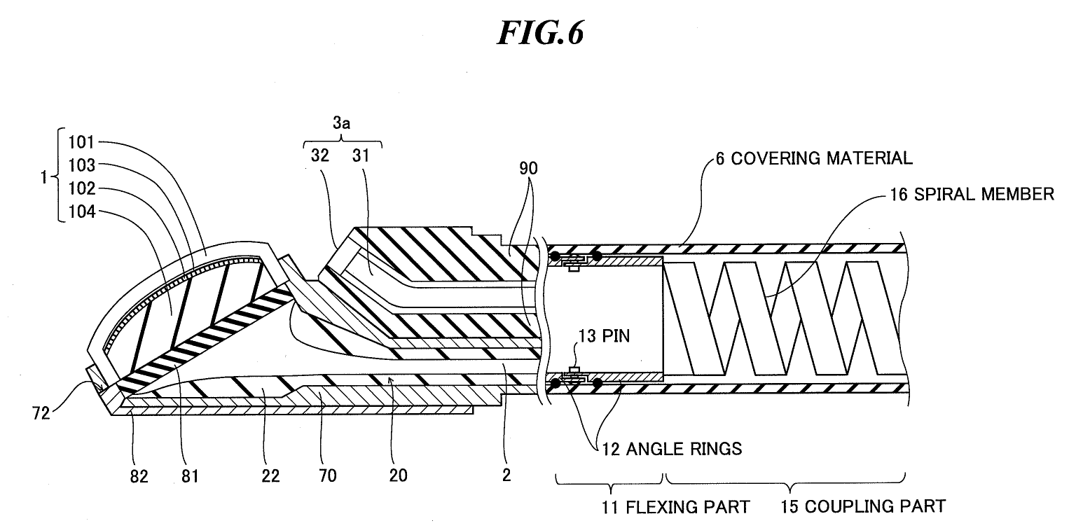

FIG. 6 is a sectional view for explanation of a configuration of an ultrasonic endoscope according to the third embodiment of the present invention, and corresponds to FIG. 3 in the first embodiment. The ultrasonic endoscope according to the embodiment has the same configuration as that of the ultrasonic endoscope according to the first embodiment except that the exterior member 70 is formed of a material having high heat conductivity (e.g., stainless steel such as SUS 304). In the embodiment, the exterior member 70 preferably has a coefficient of thermal conductivity equal to or more than 10 W/(m·K). According to a simulation, when the coefficient of thermal conductivity of the exterior member 70 is 10 W/mK in the embodiment, for example, the temperature rise of the surface of the acoustic lens 101 can be reduced by 15% compared to the case of the first embodiment. In FIG. 6, the exterior member 70 is formed of a conducting material, however, a resin having high heat conductivity may be used.

Also according to the embodiment, the same effect as that of the first embodiment can be obtained. Further, the heat that has transferred via the heat conducting member 81 can be released from the exterior member 70. Therefore, the temperature rise at the leading end of the insertion part of the ultrasonic endoscope 40 can be further suppressed.

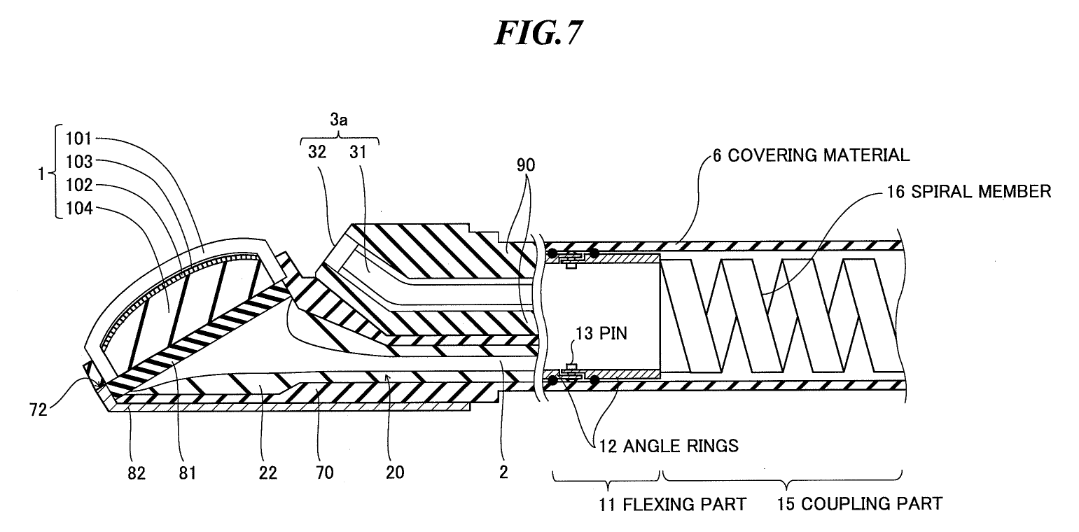

FIG. 7 is a sectional view for explanation of a configuration of an ultrasonic endoscope according to the fourth embodiment of the present invention, and corresponds to FIG. 3 in the first embodiment. The ultrasonic endoscope according to the embodiment has the same configuration as that of the first embodiment except that the heat radiating member 82 does not enter the opening 72 and the end of the heat conducting member 81 enters the opening 72 and is connected to the heat radiating member 82. Also according to the embodiment, the same effect as that of the first embodiment can be obtained.

In the above-mentioned respective embodiments, it is not necessary that the ultrasonic transducer 102 has a structure formed by stacking plural piezoelectric material layers, but may have a single piezoelectric material layer. Further, no imaging part 3 or light guide part 3a for optical observation of the object may be provided.

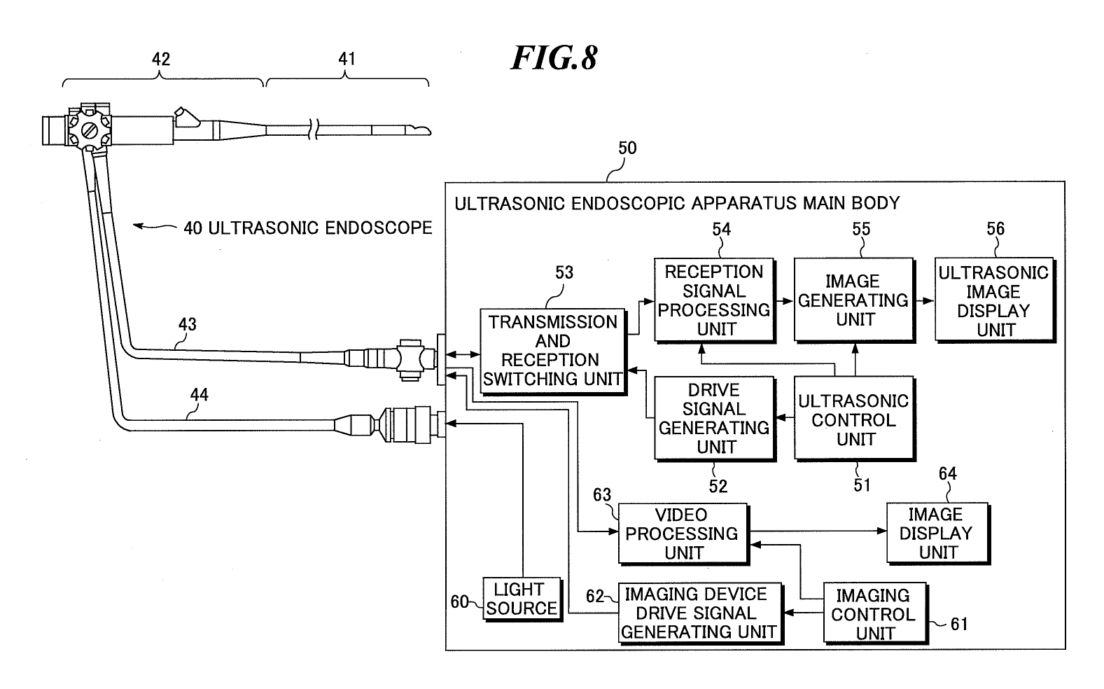

FIG. 8 shows an ultrasonic endoscopic apparatus including the ultrasonic endoscope and the ultrasonic endoscopic apparatus main body according to the respective embodiments of the present invention. The plural ultrasonic transducers contained in the ultrasonic transducer part 1 (FIG. 3) are electrically connected to the ultrasonic endoscopic apparatus main body 50 by the plural shield lines via the insertion part 41, the operation part 42, and the connecting cord 43. Those shield lines transmit drive signals generated in the ultrasonic endoscopic apparatus main body 50 to the respective ultrasonic transducers and transmit reception signals outputted from the respective ultrasonic transducers to the ultrasonic endoscopic apparatus main body 50.

The ultrasonic endoscopic apparatus main body 50 includes an ultrasonic control unit 51, a drive signal generating unit 52, a transmission and reception switching unit 53, a reception signal processing unit 54, an image generating unit 55, an ultrasonic image display unit 56, a light source 60, an imaging control unit 61, an imaging device drive signal generating unit 62, a video processing unit 63, and an image display unit 64.

The ultrasonic control unit 51 controls imaging operation using the ultrasonic transducer part 1. The drive signal generating unit 52 includes plural drive circuits (pulsers or the like), for example, and generates drive signals to be used for respectively driving the plural ultrasonic transducers. The transmission and reception switching unit 53 switches between the output of the drive signals to the ultrasonic transducer part 1 and the input of the reception signals from the ultrasonic transducer part 1.

The reception signal processing unit 54 includes plural preamplifiers, plural A/D converters, a digital signal processing circuit or CPU, for example, and performs predetermined signal processing such as amplification, phase matching and addition, and envelope detection on the reception signals to be outputted from the plural ultrasonic transducers. The image generating unit 55 generates image data representing ultrasonic images based on the reception signals on which the predetermined signal processing has been performed. The ultrasonic image display unit 56 displays ultrasonic images based on the image data generated in this manner.

The light source 60 emits light to be used for illumination of the object. The light outputted from the light source 60 illuminates the object via the optical fiber 31 (FIG. 3) of the universal cord 44 through the illumination window 32 (FIG. 3) of the insertion part 41. The illuminated object is imaged by the imaging part 3 through the observation window 3d (FIG. 2) of the insertion part 41, and video signals outputted from the imaging part 3 are inputted to the video processing unit 63 of the ultrasonic endoscopic apparatus main body 50 via the connecting cord 43.

The imaging control unit 61 controls imaging operation using the imaging part 3. The imaging device drive signal generating unit 62 generates drive signals to be supplied to the imaging part 3. The video processing unit 63 generates image data based on the video signals received from the imaging part 3. The image display unit 64 receives the image data from the video processing unit 63 and displays images of the object.

1. An ultrasonic endoscope comprising:
an ultrasonic transducer part having plural ultrasonic transducers;
an exterior member for holding said ultrasonic transducer part;
an opening formed in said exterior member;
a heat conducting member arranged inside of said exterior member and connected to said ultrasonic transducer part; and
a heat radiating member provided on an outer surface of said exterior member and connected to said heat conducting member via said opening.

2. The ultrasonic endoscope according to claim 1, wherein a part of said heat radiating member is located within said opening and connected to said heat conducting member.

3. The ultrasonic endoscope according to claim 1, wherein a part of said heat radiating member is located within said opening and connected to said exterior member.

4. The ultrasonic endoscope according to claim 1, wherein said heat radiating member is electrically insulated from said ultrasonic transducer part.

5. The ultrasonic endoscope according to claim 4, wherein at least one of said heat conducting member and said heat radiating member has an electrical insulation property.

6. The ultrasonic endoscope according to claim 1, wherein each of said heat conducting member and said heat radiating member has a coefficient of thermal conductivity not less than 10 W/(m·K).

7. The ultrasonic endoscope according to claim 6, wherein said heat radiating member is formed of stainless steel.

8. The ultrasonic endoscope according to claim 1, wherein:
said ultrasonic transducer part has a backing material;
said heat conducting member is connected to said backing material; and
said backing material is formed of a material including mixture of an electrically insulating base material and a filler having higher heat conductivity than the base material.

9. The ultrasonic endoscope according to claim 1, wherein said ultrasonic transducer has plural piezoelectric material layers stacked via electrode layers.

10. The ultrasonic endoscope according to claim 1, wherein said exterior member has a coefficient of thermal conductivity not less than 10 W/(m·K).

11. The ultrasonic endoscope according to claim 10, wherein said exterior member is formed of stainless steel.

12. The ultrasonic endoscope according to claim 1, further comprising:
signal lines connected to said ultrasonic transducer part for transmitting drive signals for said ultrasonic transducers;
a signal line holding part provided within said exterior member for holding said signal lines and having an end connected to said heat conducting member; and
a heat conducting filler filling inside of said signal line holding part.

13. The ultrasonic endoscope according to claim 12, wherein said heat conducting filler has a coefficient of thermal conductivity not less than 2 W/(m·K).
